(19) United States
(12) Patent Application Publication
(10) Pub. No.: US 2007/0299462 A1
(43) Pub. Date: Dec. 27, 2007

(54) Nasolacrimal system irrigation and dilatation tool

(76) Inventor: Bruce B. Becker, Malibu, CA (US)

Correspondence Address: CHARMASSON, BUCHACA & LEACH, LLP, 1545 HOTEL CIRCLE SOUTH, SUITE 150, SAN DIEGO, CA, 92108-3426, US

(21) Appl. No.: 11/473,445

(22) Filed: Jun. 23, 2006

(51) Int. Cl. A61M 29/00

(52) U.S. Cl. 606/192; 606/199

(57) ABSTRACT

A multi-functional surgical tool for the treatment of nasolacrimal stenosis or obstruction combines two elongated conduits, one of which is terminated at a distal end by an inflatable balloon. The other conduit is used for either irrigating a tracer fluid in a vessel that has been enlarged by inflation of the balloon, or for suctioning blood or debris loosened by the introduction and inflation of the tool. The tool is particularly useful to perform dacryocystoplasty (DCP) and dacryocystorhinostomy (DCR). Retrieval of the tracer fluid in the nasal cavity provides confirmation the all obstructions have been penetrated without false passage of the catheter through the wall of the lacrimal network. The tool avoids repeated insertion and withdrawal of the balloon catheter alternated with injection of the tracer fluid by means of another instrument.

### FIELD OF THE INVENTION

The present invention relates to devices used for normalizing the flow of fluid in tubular organs of human bodies that have been injured by a disease or an accident. More specifically, the invention relates to catheters used in treating nasolacrimal duct or canalicular stenosis, obstruction, lacerations or other traumas.

### BACKGROUND

The orbital portion of the lacrimal gland is located in the superotemporal orbit and the palpebral portion of the lacrimal gland is located on the posterior surface of the superotemporal upper lid. The lacrimal gland produces the aqueous portion of the tear film. Ductules from the orbital portion of the lacrimal gland pass through the adjacent palpebral lacrimal gland to empty in the superior conjunctival cul-de-sac. Smaller accessory lacrimal glands in the upper and lower lids also contribute to tear production. The tears bathe the surface of the eye and then drain into the puncta and canaliculi in the medial upper and lower lids. The superior and inferior canaliculi join as the short common canaliculus. The tears flow from the superior and inferior canaliculi through the common canaliculus, into the lacrimal sac, and down the nasolacrimal duct into the nose.

The canaliculi can become obstructed or stenotic, or damaged as a result of a congenital or acquired obstruction, including trauma such as laceration, inflammation, side effects of chemotherapy, or the obstruction can be idiopathic. When the upper and lower canaliculi or the common canaliculus become obstructed, tears can no longer drain from the surface of the eye through the lacrimal system into the nose. The tears well up in the eye as a result, and run down the face. The excess tears blur the vision and the patient has to constantly dab the eye.

When the nasolacrimal duct is obstructed, tears stagnate in the lacrimal sac and bacteria multiply therein leading to an infection of the lacrimal sac in many patients. The result is a painful enlargement of the lacrimal sac swollen with pus, and a discharge over the eye.

The nasolacrimal duct can be obstructed on a congenital basis (2% to 6% of newborns). The acquired form of nasolacrimal duct obstruction can result from trauma, sarcoidosis or other diseases, but most commonly is idiopathic.

A congenital nasolacrimal duct obstruction often resolves spontaneously. If it does not resolve before one year of age, then probing is performed. This is usually successful, if not balloon catheter dilatation is performed. Balloon catheter dilatation of the nasolacrimal duct or balloon catheter dacryocystoplasty (DCP) is also used as a primary procedure in children over twelve months of age, because the success rate of probing declines over a year of age. The procedure is also practiced in some case of acquired nasolacrimal duct obstruction.

When treating nasolacrimal duct obstruction with balloon catheter dilatation, a deflated balloon catheter is introduced through a punctum into one of the canaliculi, the common canaliculus, the lacrimal sac, and down the nasolacrimal duct into the nose. The surgeon must then confirm that the catheter has penetrated all obstructions and entered the nasal cavity. He must also insure that the catheter has not opened a false passage by piercing through the wall of the nasolacrimal duct instead of going down the duct into the nose.

Confirmation of the presence of the catheter into the nasal cavity is rendered difficult by the anatomy of the area. The nasolacrimal duct empties into the nose on the lateral nasal wall beneath the inferior turbinate. It is often very difficult if not impossible to observe the nasolacrimal duct and the entry of the catheter into the nose cavity. Edema of the inferior turbinate and nasal mucosal bleeding can obstruct visualization. The surgeon usually introduces a metal instrument through the external naris into the nose and tries blindly to touch the tip of the balloon catheter until a contact between the instrument and the catheter is felt. Alternatively, the surgeon may attempt to visualize the catheter with an endoscope or a headlight; a procedure with which many ophthalmic surgeons are not very familiar.

The balloon catheter is then connected to an inflation device, and its expansion dilates the duct. After deflation the catheter is pulled out of the nasolacrimal network. A syringe equipped with a short cannula is used to irrigate a tracer fluid through the lacrimal system into the nose. Recovery of some of the fluid in the nose by means of a suction device confirms that the lacrimal system is patent. If no tracer fluid is recovered, the balloon catheter has to be reintroduced in a slightly different orientation or pushed further down the nasolacrimal duct, and inflation repeated. After which a new irrigation confirming procedure must be performed. These repeated procedures often cause multiple traumas to the lacrimal drainage system.

The treatment of nasolacrimal duct obstruction in adults usually involves the creation of a new passage from the lacrimal sac directly into the nasal cavity according to a procedure called balloon catheter dacrocystorhinostomy (DCR) without attempting to dilate the nasolacrimal duct. That procedure involves a greater amount of bleeding that the balloon catheter DCP. In addition, the anterior middle turbinate may obstruct the outlet formed by the balloon DCR. Resection of the middle turbinate may be necessary leading to additional bleeding. The surgeon may extract the blood with a suction device through the nasal cavity and use an endoscope and attempt visualization with an endoscope. However this requires the surgeon to handle another instrument in addition to the endoscope. The suction may not be adequate to allow good visualization of the surgical site.

The instant invention results from attempts to avoid the aforesaid problems and provide more efficient, simpler and safer procedures in the treatment of nasolacrimal duct obstructions.

### SUMMARY

It is an object of the invention to better treat obstructions in the nasolacrimal system. This and other objects are achieved by a multiconduit catheter.

In some embodiments there is provided a dilating balloon catheter designed to be inserted into a patient nasolacrimal system which incorporates an irrigation catheter that allows immediate injection of a tracer fluid without having to retract the catheter. In some embodiments, retrieval of the fluid in the nasal cavity provides confirmation that all obstructions have been penetrated without false passage of the catheter through the wall of the lacrimal network. The combination catheter can also be run through a new passageway pierced with a probe through the inferomedial wall of the lacrimal sac and the lateral nasal wall.

In some embodiments, the multi-functional surgical tool for the treatment of nasolacrimal duct obstruction comprises a first elongated tubular conduit having proximal and distal ends, a second elongated tubular conduit having proximal and distal extremities. The second conduit can be parallelly coupled to the first conduit. In some embodiments, an inflatable balloon is provided at a distal end of the first conduit. The surgical tool can be sized to be introduced into a patient's nasolacrimal duct through one of the patient's canaliculi.

In some embodiments, the coupled conduits have a total maximum cross-sectional dimension ranging between about 1.0 millimeter and 2.5 millimeters, and can be coaxial, wherein the second conduit comprises a median portion running inside the first conduit and balloon, a proximal section exiting through an opening in the first conduit, and a distal section exiting through an aperture beyond the balloon.

In some embodiments, the opening in the first conduit is sealed around the proximal section and the aperture is sealed around the distal section of the second conduit.

In some embodiments, the tool further comprises a first connector at the proximal end of the first conduit and a second connector at the proximal extremity of the second conduit.

In some embodiments, the tool also comprises an irrigation device connected to the second connector. Alternately, a suction device may be connected to the second connector.

In some embodiments, the tool further comprises an inflating device connected to the first connector.

In some embodiments, there is provided a modified version of the tool where the second conduit has an angled distal part.

In some embodiments, there is provided a surgical tool which combines a balloon catheter and an irrigation conduit coupled together in an elongated instrument which is sized to be manually introduced into a patient's nasolacrimal network, wherein an inflatable balloon and a discharge outlet of the irrigation conduit are located at the distal end of the instrument.

In some embodiments, there is provided a method for treating an obstruction in a patient nasolacrimal system, which comprises: inserting into said system a tool combining a balloon catheter and an irrigation catheter, joined parallelly to each other; inflating said balloon catheter; and injecting a tracer fluid through said irrigation catheter.

In some embodiments, the method further comprises detecting an amount of said tracer fluid beyond said obstruction.

In some embodiments, the method further comprises successively inflating and deflating said balloon catheter, and performing a plurality of said injecting steps.

In some embodiments, the method further comprises suctioning blood out of the nasolacrimal system through the irrigation catheter, and injecting medication in the system through said irrigation catheter.

In some embodiments, the method further comprises piercing a passageway through the patient's inferomedial wall with a probe and running the tool through said passageway.

### BRIEF DESCRIPTION OF THE DRAWING

FIG. 1 is a cross-sectional view of an exemplary embodiment of a surgical tool according to the invention;

FIG. 2 is a diagrammatical illustration of the use of the tool in a DCP;

FIG. 3 is a diagrammatical illustration of the use of the tool in a DCR; and

FIG. 4 is a diagrammatical illustration of the use of the tool in a transnasal DCR.

### DESCRIPTION OF THE PREFERRED EMBODIMENT

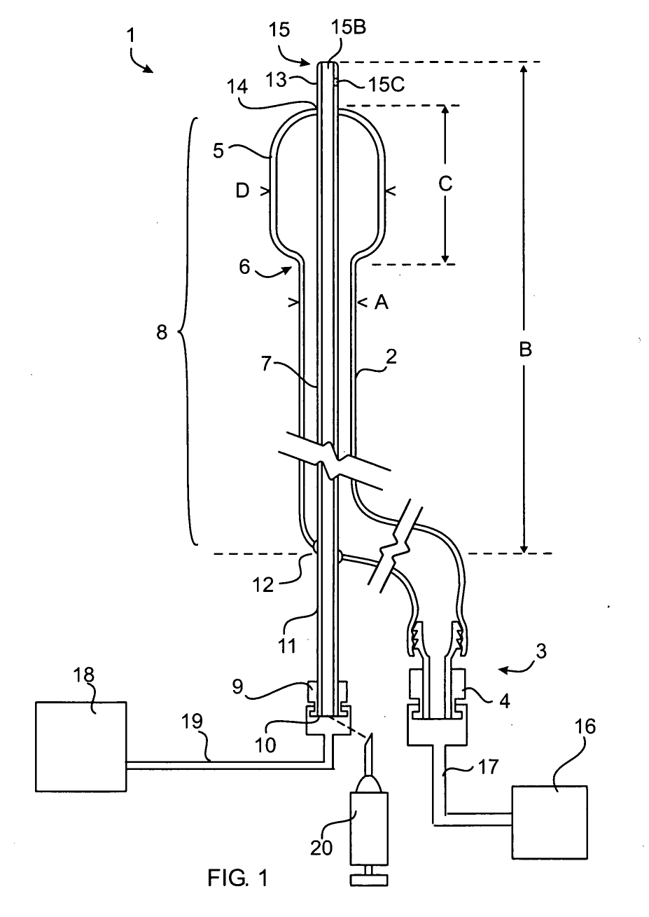

Referring now to the drawing, there is shown in FIG. 1 a multi-functional surgical tool 1 for the treatment of a nasolacrimal obstruction. The tool comprises a first elongated conduit 2 having at a proximal end 3 a luer-lock 4 or other type of connector, and an inflatable balloon 5 at a distal end 6. A second elongated conduit 7 is parallelly coupled to the first conduit. In this case, a median section 8 of the second conduit runs near coaxially within the first conduit 2. A second luer-lock or other type connector 9 is attached to the proximal extremity 10 of the second conduit. The proximal section 11 of the second conduit exits the first conduit through an opening 12 in the wall of the first conduit. A distal portion 13 of the second conduit exits through an aperture 14 beyond but near the inflatable balloon 5. While the distal end 6 of the first conduit is closed by the inflatable balloon 5, the second conduit has a blunt distal end 15 and an axial port 15B. In addition, one or more radial ports 15C through a sidewall of the distal portion 13 and thereby near the distal end 15 provide a fluid passageway should the axial port be blocked. The opening 12 and aperture 14 are hermetically sealed around the second conduit.

It would be an obvious modification to run the first conduit within the second one with the inflatable balloon being positioned beyond the distal end of the second conduit. The two conduits could also run contiguously rather than coaxially.

The tool is sized to be introduced into a patient's nasolacrimal duct through one of said patient's canaliculi. The total cross-sectional diameter A of the combined conduits has a maximum dimension of between about 0.1 and 10 millimeters, and typically about 1 millimeter for the present embodiment and about 2.5 millimeters for a transnasal embodiment described below. The total insertable length B of the tool may be within range from about 1 to 50 centimeters, and is typically about 15 centimeters. The inflatable balloon 5 has a length C between about 0.5 and 5 centimeters, and an inflated cross-sectional diameter D of up to about 2 centimeters. The balloon portion is preferably made of a resiliently expandable synthetic material such as polyethylene terephthalate (PET), latex, silicon or other elastomeric material. It can also be made of nylon, polyurethane, polyvinyl chloride, cross-linked polyethylene, polyolefins, HPTFE, HPE, HDPE, LDPE, EPTFE, and block polymers. The remainder of the first conduit is preferably made of polyethylene terephthalate (PET).

The second conduit 7 is preferably made of an hard but flexible material such as stainless steel and has enough rigidity to be pushed through obstructions in the nasolacrimal network, but is flexible enough to bend around small curves. Other metals and alloys such as titanium, silver, aluminum, bronze, brass, and synthetics like Kevlar and Nitinol may also be suitable.

During use, the first connector 4 at the proximal end of the first conduit is connected to an inflation device 16 by means of an appropriate connecting tube 17.

The second connector 9 at the proximal end of the second conduit is connected to a suction device 18 through an appropriate connecting tube 19 or, at other times, to a syringe 20 or other irrigation devices. In this way the port can act as an entry port for suctioned material or as an exit port for irrigation fluid.

The above-described surgical tool can be used in a variety of surgical interventions as explained below.

In a balloon catheter DCP procedure, the surgeon begins dilating the punctum with a punctal dilator before inserting a probe through the punctum and canaliculus down to the lacrimal sac. A barrier is felt when the probe encounters the medial lacrimal sac wall and lacrima fossa. The probe is then retracted about 0.5 millimeters and is tilted about 90 degrees into alignment with the nasolacrimal duct. The probe is pushed down the nasolacrimal duct and into the nasal cavity, then removed.

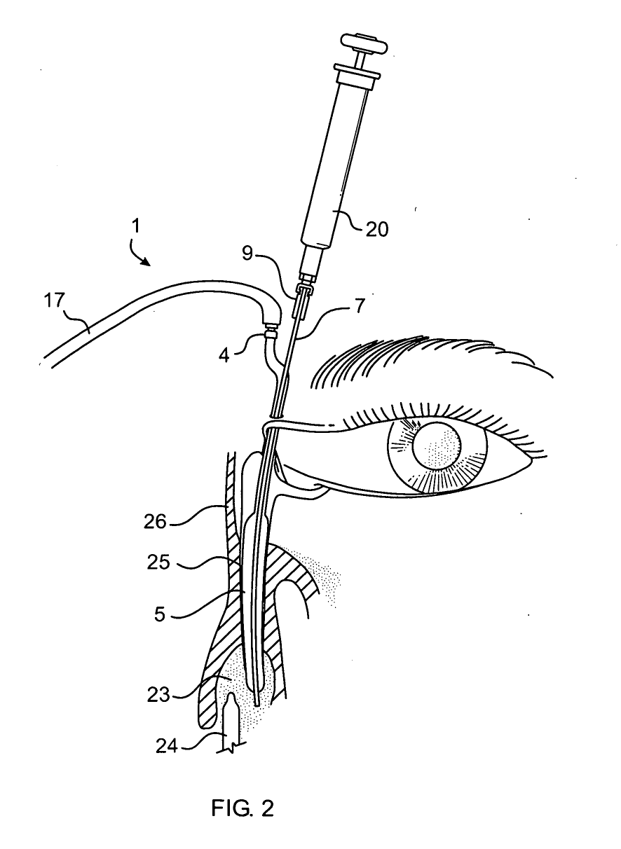

The tool 1, with the balloon 5 deflated, is inserted in the same manner as the probe down to the nasal cavity 23 as illustrated in FIG. 2. The syringe 20 can be connected directly to the luer-lock connector 9 of the second conduit 7. Alternatively, the flexible tube 19 is connected at one end to the luer-lock connector 9 and at the other end to the syringe 20 loaded with fluorescein stained fluid or any other tracing fluid. The fluid is injected to irrigate through the second conduit 7 into the nose. Traces of the fluid can be recovered in the nose with a suction device 24. A lack of fluid in the nose indicates that the tool has not penetrated all obstructions and reached the nose. The surgeon can then push with greater force or pull the tool slightly and drive it into the nasal cavity at a slightly different angle. Detection of the tracing fluid in the nose is a positive indication that all obstructions have been penetrated.

It should be noted that the surgeon does not have to perform the difficult and sometime impossible task of touching the tip of the tool in the nose with another metal instrument in order to confirm that the tool has duly entered the nasal cavity.

The tube 17 from the inflation device 16 is connected to the luer-lock connector 4 at the proximal end of the first conduit. An inflating fluid is sent down that conduit to inflate the balloon 5 and dilate the stenoic nasolacrimal duct 25. The balloon is deflated and pulled more proximally before a new inflation cycle is performed. The procedure is repeated as many times as it may be necessary to dilate the entire duct and the sac-duct junction. An irrigation cycle is again performed. If irrigation is not successful the tool may be pushed back before repeating the inflation procedure until tracer fluid recovery in the nose confirms that all obstructions have been corrected.

If significant bleeding occurs during the procedure, the syringe 20 is removed and the tube 19 is connected to the suction device 18 in order to remove the blood.

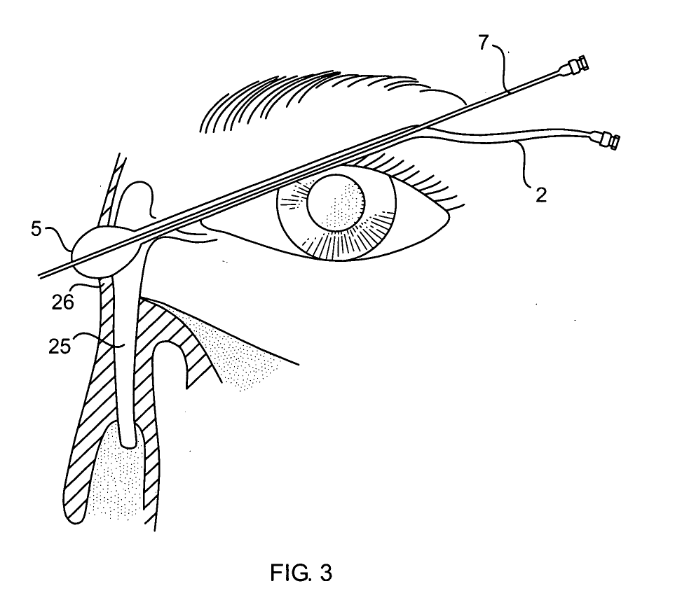

In a balloon catheter DCR, a probe is inserted as described above in connection with a DCP into the lacrimal sac. It is the pushed through the inferomedial wall of the sac, lacrimal fossa, and lateral nasal wall into the nose. The probe is visualized endoscopically. The probe is further pushed through multiple adjacent areas to enlarge the opening and push bone chips of lacrimal fossa bone and possibly ethmoid bone into the nasal cavity. The probe is withdrawn, and the tool 1 is pushed through the superior or inferior canaliculus, the common canaliculus, the lacrimal sac and through the prepared opening in the inferomedial lacrimal sac wall, lacrimal fossa, lateral nasal wall into the nasal cavity. A syringe is used to inject a tracer fluid through the second conduit 7 as shown in FIG. 3. The presence of tracer fluid in the nose is confirmed. The syringe is then replaced by the suction device 18, and blood and tissue debris are suctioned. Bleeding is usually more profuse than in a DCP and suctioning may have to be performed during the entire operation.

Inflation of the balloon 5 by the inflation device 16, through the tube 17 and the first conduit 2 is performed as described earlier including about the opening into the inferomedial wall 26 as shown in FIG. 3 and lateral nasal wall. The tool in then withdrawn.

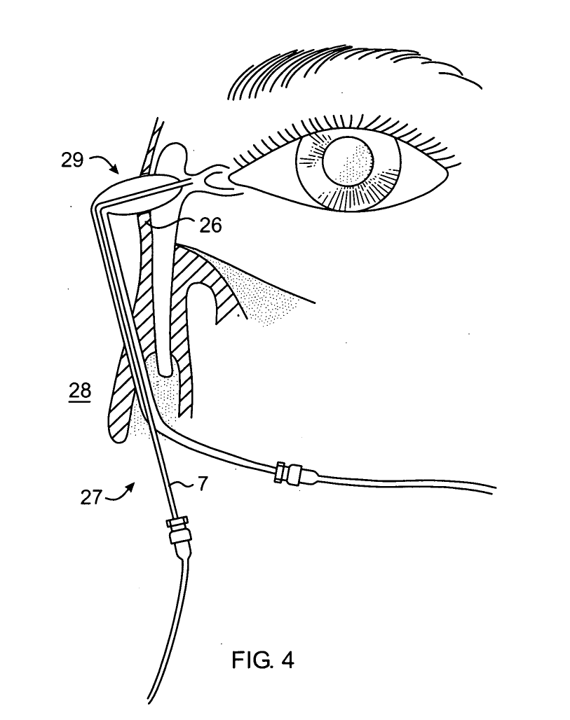

The performance of a transnasal balloon catheter DCR is performed basically in the same manner as a above, except that after probing and piercing of the inferomedial wall and lateral nasal wall, a modified tool 27 is brought up through the external naris 28 up the nasal cavity and pushed through the opening in the inferomedial wall 26 of the lacrimal sac into the lacrimal sac as shown in FIG. 4.

The modified tool has a distal segment 29 bent at an angle of between about 10 and 170 degrees, and is typically about 90 degrees.

A suction procedure is performed through the second conduit 7, then a dilatation procedure as described above. After disconnecting the suction device 18, irrigation may be performed.

The second conduit 7 can also be used at that time to deliver medications into the nasolacrimal duct.

After having removed the tool 27, the surgeon inserts a short cannula of about 1.0 centimeter in length into the punctum and canaliculus. With syringe, a tracer fluid is injected through the cannula into the nasolacrimal network. If none of the fluid is recovered in the nose, the procedure must be repeated.

In lieu of a tracer fluid, a radioopaque or isotopic solution can be injected. An x-ray or radiation detecting machine is used to confirm the proper penetration of the tool.

While the preferred embodiment of the invention has been described, modifications can be made and other embodiments may be devised without departing from the spirit of the invention and the scope of the appended claims.

What is claimed is:

1. A multi-functional surgical tool for the treatment of nasolacrimal obstruction which comprises:
a first elongated tubular conduit having proximal and distal ends;
a second elongated tubular conduit having proximal and distal extremities, said second conduit being parallelly coupled to said first conduit; and
an inflatable balloon at a distal end of said first conduit;
said surgical tool being sized to be introduced into a patient's nasolacrimal duct through one of said patient's canaliculi.

2. The tool of claim 1, wherein said second conduit comprises at least one port near said distal extremity.

3. The tool of claim 1, wherein said coupled conduits have a maximum cross-sectional dimension of between about 1.0 millimeter and 2.5 millimeters.

4. The tool of claim 3, wherein said first and second conduits are coaxial.

5. The tool of claim 4, wherein said second conduit comprises:
a median portion running inside said first conduit and balloon;
a proximal section exiting through an opening in said first conduit; and
a distal section exiting through an aperture beyond said balloon.

6. The tool of claim 5, wherein said opening is sealed around said proximal section and said aperture is sealed around said distal section.

7. The tool of claim 6, which further comprises a first connector at said proximal end and a second connector at said proximal extremity.

8. The tool of claim 7, which further comprises an irrigation device connected to said second connector.

9. The tool of claim 7, which further comprises a suction device connected to said second connector.

10. The tool of claim 7, which further comprises an inflating device connected to said first connector.

11. The tool of claim 7, wherein said second conduit has an angled distal part.

12. A surgical tool which combines a balloon catheter and an irrigation conduit coupled together in an elongated instrument sized to be manually introduced into a patient's nasolacrimal network, wherein an inflatable balloon and a discharge port of the irrigation conduit are located near the distal end of the instrument.

13. A method for treating an obstruction in a patient nasolacrimal system, which comprises:
inserting into said system a tool combining a balloon catheter and an irrigation catheter, joined parallelly to each other;
inflating said balloon catheter; and
injecting a tracer fluid through said irrigation catheter.

14. The method of claim 13, which further comprises detecting an amount of said tracer fluid beyond said obstruction.

15. The method of claim 13, which further comprises successively inflating and deflating said balloon catheter, and performing a plurality of said injecting steps.

16. The method of claim 13, which further comprises suctioning blood out of said system through said irrigation catheter.

17. The method of claim 13, which further comprises injecting medication in said system through said irrigation catheter.

18. The method of claim 13, which further comprise piercing a passageway through said patient's inferomedial wall with a probe and running said tool through said passageway.
